(19) United States
(12) Patent Application Publication
(10) Pub. No.: US 2010/0008971 A1
(43) Pub. Date: Jan. 14, 2010

(54) CHEWING COMPOSITION COMPRISING COENZYME Q10

(75) Inventors: Hideyuki Orikoshi, Osaka (JP); Yoshiaki Maeda, Osaka (JP); Taisuke Nakamura, Saitama (JP); Takumi Tokumoto, Saitama (JP); Masaki Yanazaki, Saitama (JP)

Correspondence Address: KRATZ, QUINTOS & HANSON, LLP, 1420 K Street, N.W., Suite 400, WASHINGTON, DC, 20005, US

(73) Assignees: SAN-EI GEN F.F.I., INC., TOYONAKA-SHI (JP); LOTTE CO., LTD., TOKYO (JP)

(21) Appl. No.: 12/296,586

(22) PCT Filed: Apr. 11, 2007
(86) PCT No.: PCT/JP2007/057954

§ 371 Date: Mar. 12, 2009

(30) Foreign Application Priority Data
Apr. 11, 2006 (JP) 2006-109072

(51) Int. Cl. A61K 9/68; A61P 35/00; A61P 9/10

(52) U.S. Cl. 424/440; 424/48

(57) ABSTRACT

An object of the invention is to provide a chewing composition containing Coenzyme Q₁₀ with improved release properties in the oral cavity. Another object is to provide a process for improving the release properties of Coenzyme Q₁₀ in the oral cavity.

Coenzyme Q₁₀ is encapsulated in cyclodextrin, and the resulting inclusion complex is used as a Coenzyme Q₁₀ component. The content of Coenzyme Q₁₀ in the inclusion complex is preferably low. It is preferably 40 wt % or less, and more preferably 20 wt %.

### TECHNICAL FIELD

The present invention relates to chewing compositions containing Coenzyme Q₁₀; and more particularly to chewing compositions containing Coenzyme Q₁₀ with improved release properties in the oral cavity. The present invention also relates to a method for improving the release properties of Coenzyme Q₁₀; and more particularly to a method for improving the release properties of Coenzyme Q₁₀ in the oral cavity from a chewing composition containing Coenzyme C₁₀.

### BACKGROUND ART

Coenzyme Q₁₀ is an essential component for bioenergy production, which is mostly found in the mitochondria of eukaryotic cells as an indispensable component for ATP (adenosine triphosphate) production in the human body.

Coenzyme Q₁₀ is also known to function as an excellent antioxidant in vivo, and expected to be effective for preventing diseases that can be associated with reactive oxygen species in vivo; for example, so-called lifestyle-related diseases, such as myocardial infarction, hypertension, angina and cancer. Moreover, it is also considered to be effective for preventing brain diseases such as Alzheimer, Parkinson's disease and depression; disease of the gums, muscular dystrophy, and various other diseases, and also effective for anti-aging and inhibiting obesity owing to its function of promoting metabolism.

Part of the Coenzyme Q₁₀ necessary for the body is supplied through endogenous synthesis, but otherwise is consumed from foods. While the amount of Coenzyme Q₁₀ endogenously synthesized is known to decrease with age, Coenzyme Q₁₀ can only be consumed in small amounts from foods. Consequently, the total amount of internally supplied Coenzyme Q₁₀ decreases with increased age. Therefore, for the purpose of supplementing the amount that is endogenously synthesized, external intake of Coenzyme Q₁₀ in the forms of, for example, foods and supplements, has become popular today.

Coenzyme Q₁₀ is a lipid-soluble substance. Therefore, currently-commercially-available Coenzyme Q₁₀ in oral-dosage forms are typically formulated by dispersing or dissolving a crystalline powder of Coenzyme Q₁₀ in an oil, or by filling the crystalline powder with capsules.

It is also known that when added to a food, Coenzyme Q₁₀ becomes unstable due to the influence of other additives; in particular, when coexisting with vitamins, especially vitamin E, Coenzyme Q₁₀ becomes extremely unstable (see Patent Document 1). Since vitamins are the usual ingredients of medicines, quasi drugs, foods and feed, it is necessary to take some measures to stabilize Coenzyme Q₁₀ in a preparation or formulation, in order to add it to these products.

Methods for stabilizing Coenzyme Q₁₀ are disclosed in Patent Documents 1 and 2, which comprise encapsulating Coenzyme Q₁₀ in cyclodextrin. More specifically, Patent Document 1 teaches that the encapsulation of Coenzyme Q₁₀ in cyclodextrin enables stabilization of the Coenzyme Q₁₀ even in the presence of vitamins and other additives, without decreasing the stabilities of vitamins and other ingredients used together with Coenzyme Q₁₀. Moreover, Patent Document 2 teaches that the encapsulation of Coenzyme Q₁₀ in cyclodextrin imparts Coenzyme Q₁₀ with stability against physical or chemical action, especially the action of UV light, so that the cyclodextrin inclusion complex is useful as an ingredient of cosmetic formulations. Cyclodextrin is hence a substance widely used as a protective coating for susceptible molecules in the pharmaceutical, cosmetic and food fields.
Patent Publication 1: Japanese Unexamined Patent Application Publication No. 2005-2005
Patent Publication 2: Japanese Unexamined Patent Application Publication No. 2002-104922

### DISCLOSURE OF THE INVENTION

### Problems to be Solved by the Invention

An object of the invention is to provide a chewing composition containing Coenzyme Q₁₀ with improved release properties in the oral cavity. Another object of the invention is to provide a method for improving the release properties of Coenzyme Q₁₀; and more particularly to a method for improving the release properties of Coenzyme Q₁₀ in the oral cavity from a chewing composition containing Coenzyme Q₁₀.

### Means for Solving the Problem

The inventors conducted extensive research to solve the aforementioned problems, and found that when Coenzyme Q₁₀ is encapsulated in cyclodextrin, the release properties of the Coenzyme Q₁₀ in the oral cavity are improved. The inventors also found that the release properties of Coenzyme Q₁₀ are remarkably enhanced by lowering, rather than increasing, the proportion of Coenzyme Q₁₀ added to cyclodextrin, and ascertained that the use of a cyclodextrin inclusion complex containing a moderate proportion of Coenzyme Q₁₀ allows the preparation of chewing compositions (such as chewing gums) containing Coenzyme Q₁₀ with excellent release properties in the oral cavity. The present invention was thus accomplished based on these findings.

Embodiments included in the invention are as set forth below:

### I. Chewing Composition Containing Coenzyme Q₁₀ Inclusion Complex

(1-1): A chewing composition comprising a Coenzyme Q₁₀ inclusion complex in which Coenzyme Q₁₀ is encapsulated in cyclodextrin.
(1-2): The chewing composition according to (I-1), wherein the Coenzyme Q₁₀ inclusion complex comprises 1 to 10 wt % of Coenzyme Q₁₀.
(1-3): The chewing composition according to (I-1) or (I-2), wherein the content of Coenzyme Q₁₀ is 1 to 80 parts by weight to 100 parts by weight of cyclodextrin.
(I-4): The chewing composition according to any one of (I-1) to (I-3), wherein the content of the Coenzyme Q₁₀ inclusion complex is 0.08 to 25 wt % in 100 wt % of the chewing composition.
(I-5): The chewing composition according to any one of (I-1) to (I-4), wherein the content of the Coenzyme Q₁₀ inclusion complex is such that the proportion of Coenzyme Q₁₀ in 100 wt % of the chewing composition is 0.01 to 10 wt %.
(I-6): The chewing composition according to any one of (I-1) to (I-5), wherein the cyclodextrin is γ-cyclodextrin.
(I-7): The chewing composition according to any one of (I-1) to (I-6), further comprising a gum base.
(I-8): The chewing composition according to (I-7), which is a chewing gum.
(I-9): The chewing composition according to any one of (I-1) to (I-8), wherein the chewing composition is covered with a sugar coating comprising the Coenzyme Q₁₀ inclusion complex.

### II. Method for Improving Release Properties of Coenzyme Q₁₀

(II-1): A method for improving the release properties of Coenzyme Q₁₀, comprising encapsulating Coenzyme Q₁₀ in cyclodextrin.
(II-2): A method for improving the release properties of Coenzyme Q₁₀ from a chewing composition containing a Coenzyme Q₁₀ component, the process comprising adding to the composition a Coenzyme Q₁₀ inclusion complex in which Coenzyme Q₁₀ is encapsulated in cyclodextrin as the Coenzyme Q₁₀ component.
(II-3): The method according to (II-2), wherein the Coenzyme Q₁₀ inclusion complex comprises 1 to 40 wt % of Coenzyme Q₁₀.
(II-4) : The method according to (II-2) or (II-3), wherein the content of Coenzyme Q₁₀ in the Coenzyme Q₁₀ inclusion complex is 1 to 80 parts by weight to 100 parts by weight of cyclodextrin.
(II-5): The method according to any one of (II-2) to (II-4), wherein the content of the Coenzyme Q₁₀ inclusion complex is 0.08 to 25 wt % in 100 wt % of the chewing composition.
(II-6): The method according to any one of (II-2) to (II-5), comprising adding the Coenzyme Q₁₀ inclusion complex in an amount such that the content of Coenzyme Q₁₀ is 0.01 to 10 wt % in 100 wt % of the chewing composition.
(II-7): The method according to any one of (II-1) to (II-6), wherein the cyclodextrin is γ-cyclodextrin.
(II-8): The method according to any one of (II-2) to (II-7), wherein the chewing composition further comprises a gum base.
(II-9): The method according to (II-8), wherein the chewing composition is a chewing gum.
(II-10): The method according to any one of (II-2) to (II-9), wherein the chewing composition is covered with a sugar coating comprising the Coenzyme Q₁₀ inclusion complex.
(II-11): The method according to any one of (II-2) to (II-11), which is a process for improving the release properties of Coenzyme Q₁₀ in the oral cavity from a chewing composition containing Coenzyme Q₁₀.

### III. Use

(III-1): Use of a Coenzyme Q₁₀ inclusion complex in which Coenzyme Q₁₀ is encapsulated in cyclodextrin, for the preparation of a chewing composition containing Coenzyme C₁₀.
(III-2): The use according to (III-1), wherein the chewing composition is a chewing gum.
(III-3): The use according to (III-1) or (III-2), wherein the Coenzyme Q₁₀ inclusion complex comprises 1 to 40 wt % of Coenzyme Q₁₀.
(III-4): The use according to any one of (III-1) to (III-3), wherein the content of Coenzyme Q₁₀ in the Coenzyme Q₁₀ inclusion complex is 1 to 80 parts by weight to 100 parts by weight of cyclodextrin.
(III-5): The use according to any one of (III-1) to (III-4), wherein the content of the Coenzyme Q₁₀ inclusion complex is 0.08 to 25 wt % in 100 wt % of the chewing composition.
(III-6): The use according to any one of (III-1) to (III-5), wherein the Coenzyme Q₁₀ inclusion complex is used in an amount such that the content of Coenzyme Q₁₀ is 0.01 to 10 wt % in 100 wt % of the chewing composition.
(III-7): The use according to any one of (III-1) to (III-6), wherein the cyclodextrin is γ-cyclodextrin.
(III-8): The use according to any one of (III-1) to (III-7), wherein the chewing composition is covered with a sugar coating comprising the Coenzyme Q₁₀ inclusion complex.

### Effects of the Invention

The present invention provides a Coenzyme Q₁₀ inclusion complex containing Coenzyme Q₁₀ with excellent release properties; more particularly, a Coenzyme Q₁₀ inclusion complex that exhibits improved release properties with a moderate content of Coenzyme Q₁₀. The use of the Coenzyme Q₁₀ inclusion complex allows the preparation of chewing compositions (such as chewing gums) capable of efficiently releasing large amounts of Coenzyme Q₁₀ in the oral cavity. In accordance with the invention, the use of a large amount of a Coenzyme Q₁₀ inclusion complex with a low Coenzyme Q₁₀ content, rather than the use of a Coenzyme Q₁₀ inclusion complex with a high Coenzyme Q₁₀ content, enables a higher intake of Coenzyme Q₁₀, even though the content of Coenzyme Q₁₀ in the final chewing composition is the same in both cases.

### BEST MODE FOR CARRYING OUT THE INVENTION

### (1) Chewing Composition Containing Coenzyme Q₁₀

The present invention relates to a chewing composition containing Coenzyme Q₁₀. The chewing composition of the invention is a composition chewed in the mouth, or a composition consumed by chewing it in the mouth. Specific examples of such compositions include foods such as chewing gums, bubble gums and other gums (including stick gums, tablet gums and sugar-coated gums), gummies, nougat, etc.; medicines such as masticatories, chewable tablets, granules, troches, buccal tablets, tooth pastes, etc.; and quasi drugs such as tooth pastes, etc. The chewing composition is preferably a chewing gum. The chewing composition is also preferably coated with sugar.

The chewing composition of the invention comprises Coenzyme Q₁₀ in a form such that the Coenzyme Q₁₀ is encapsulated in cyclodextrin (in the form of a Coenzyme Q₁₀ inclusion complex). The proportion of Coenzyme Q₁₀ in the inclusion complex can be suitably selected from the range of 1 to 80 wt %; the upper limit being preferably 40 wt %, more preferably 20 wt %, and still more preferably 10 wt.%; and the lower limit being preferably 1 wt %, more preferably 2 wt %, and still more preferably 5 wt %. More specifically, the range is preferably from 1 to 40 wt %, more preferably 2 to 20 wt %, and still more preferably 2 to 10 wt %.

Cyclodextrin for use in the Coenzyme Q₁₀ inclusion complex is a host molecule for the inclusion complex, which composed of six, seven, eight or more glocose residues α-1,4-linked in a ring structure. Typically known are α-, β-, γ- and δ-cyclodextrins. While any of α-, β-, γ- and δ-cyclodextrins, as well as their derivatives are usable in the invention, β- and γ-cyclodextrins are preferable. γ-Cyclodextrin is more preferable.

Coenzyme Q₁₀ for use in the invention is an ubiquinone, in which the number (n) of isoprenoid side chains of 2,3-dimethoxy-5-methyl-6-polyprenyl-1,4-benzoquinone is 10. Coenzyme Q₁₀ is a lipid-soluble substance, which is also referred to as ubidecarenone, CoQ₁₀, or coenzyme UQ₁₀. Coenzyme Q₁₀ for use in the invention may be of any origin or may be prepared in any process, as long as it can be taken as a medicine, or is edible as a food. For example, Coenzyme Q₁₀ can be prepared by the methods disclosed in Japanese Unexamined Patent Application Publications Nos. 1979-122795 and 1980-19006.

Various known processes can be used for encapsulating Coenzyme Q₁₀ in cyclodextrin. Examples of such known processes include kneading Coenzyme Q₁₀ and cyclodextrin in water, mixing and pulverizing Coenzyme Q₁₀ and cyclodextrin, freeze-drying, use of a saturated solution, and co-precipitation (see, for example, WO 01/054730 and Journal Acta Poloniae Pharmaceutics 52 (5s), pp. 379-386, 1995).

During the preparation of the Coenzyme Q₁₀ inclusion complex, other ingredients, in addition to Coenzyme Q₁₀, may be encapsulated in cyclodextrin. Examples of such ingredients include flavoring agents such as l-menthol, dl-menthol, orange essential oils and the like; vitamins, such as vitamin A, vitamin D, vitamin E, vitamin K and the like; minerals such as calcium, potassium, magnesium and the like; yeasts and extracts thereof; plant extracts such as garlic, green tea and other extracts; and functional raw materials such as carotinoides, α-lipoic acid, sterols, isoflavone, collagen, peptides, amino acids, L-carnitine, γ-aminobutyric acid, taurine, glucuronic acid salt, soy isoflavone, lecithin, hyaluronic acid, chondroitin sulfate, saturated or unsaturated fatty acids and esters thereof, vegetable fiber and the like.

The Coenzyme Q₁₀ inclusion complex thus prepared exhibits significantly improved release properties of Coenzyme Q₁₀ at a relatively low content of Coenzyme Q₁₀, without the need to contain a high content of Coenzyme Q₁₀. The upper limit of the content of Coenzyme Q₁₀ is preferably 40 wt %, more preferably 20 wt %, and still more preferably 10 wt %; and the lower limit is preferably 1 wt %, more preferably 2 wt %, and still more preferably 5 wt %. More specifically, the range is preferably from 1 to 40 wt %, more preferably 2 to 20 wt %, and still more preferably 2 to 10 wt %.

Although the proportion of Coenzyme Q₁₀ to cyclodextrin in the Coenzyme Q₁₀ inclusion complex is not limited in the conditions described hereinabove, it is typically 1 to 80 parts by weight to 100 parts by weight of cyclodextrin. It is preferably 2 to 40 parts by weight, and more preferably 5 to 20 parts by weight.

The chewing composition may include Coenzyme Q₁₀ in the form of a Coenzyme Q₁₀ inclusion complex as explained above. The chewing composition may also include conventional ingredients depending on the type and shape of the chewing composition, and may be prepared according to a conventional process. While the proportion of the Coenzyme Q₁₀ inclusion complex in the chewing composition is not limited, it may suitably be selected from a typical range of 0.08 to 25 wt % per 100 wt % of the chewing composition. The range is preferably from 1 to 10 wt %, and more preferably 2 to 5 wt %. The content of the Coenzyme Q₁₀ inclusion complex in the chewing composition, when calculated as the Coenzyme Q₁₀ content, is preferably from 0.01 to 10 wt %, more preferably 0.1 to 4 wt %, and still more preferably 0.2 to 2 wt %, per 100 wt % of the chewing composition.

Examples of ingredients added to the chewing composition other than the Coenzyme Q₁₀ inclusion complex include gum bases, thickeners, brighteners, emulsifiers, spice extracts, saccharides, oil and fats, flavoring agents, sweeteners, acidulants, colorants, softeners, antioxidants, seasonings, fortifiers and the like.

A gum base may comprise components usually contained in chewing gums. Examples of such components include various components recognized as gum-base food additives and mixtures thereof; more specifically, urushi wax, ester gum, elemi resin, ozokerite, opopanax resin, carnauba wax, candelilla wax, guaiacum wood resin, guayule, gutta hang kang, gutta percha, glycerine fatty acid esters, copaiba balsam, copal resin, rubber, rubber decomposites, rice bran wax, vinyl acetate resin, sugarcane wax, sandarac resin, shellac wax, jelutong, sucrose fatty acid esters, sorva, sorbitan fatty acid esters, sorvinha, talc, calcium carbonate, damar resin, chicle, chilte, tunu, depolymerized natural, rubber, niger gutta, olibanum, paraffin wax, propylene glycol fatty acid esters, powdered pulp, powdered chaff, Venezuelan chicle, benzoin gum, jojoba wax, polyisobutylene, polybutene, microcrystalline wax, mastic, massaranduba chocolate, massaranduba balata, bees wax, myrrh, Japan wax, montan wax, lanolin, tricalcium phosphate, calcium monohydrogen phosphate, leche de vaca, rosidinha, rosin and the like. When a gum base is present, the content of the gum base is typically 10 to 35 wt %, and preferably 20 to 30 wt %, of the chewing composition.

Examples of sweeteners include monosaccharides, disaccharides, oligosaccharides, sugar alcohols and intense sweeteners. Examples of preferable sweeteners include saccharides such as cane sugar, fructose, liquid sugar, glucose, oligosaccharides and the like; intense sweeteners such as aspartame, sucralose, acesulfam K, thaumatin, stevia, alitame, neotame, xylitol, saccharin salts, glycyrrhizin and the like.

Examples of colorants include β-carotene, carotenoid color, red pepper color, annatto extract, madder color, orange color, cacao color, gardenia color, chlorophyll, shikon color, erythrosine, tartrazine, onion color, tomato color, marigold color, lutein, caramel color, copper chlorophyll, grape skin color, riboflavin, riboflavin 5′-phosphate sodium and the like.

Examples of thickeners include gum arabic, carrageenan, karaya gum, carboxymethyl cellulose calcium, xanthan gum, cyamoposis gum, glucosamine, gellant gum, thalla gum, dextran, tragacanth gum, seed gum, pullulan, pectin, rhamsan gum and the like.

Examples of acidulants include adipic acid, itaconic acid, citric acid, monopotassium citrate, tripotassium citrate, trisodium citrate, glucono-delta-lactone, gluconic acid, potassium gluconate, sodium gluconate, succinic acid, monosodium succinate, disodium succinate, sodium acetate, DL-tartaric acid, L-tartaric acid, disodium DL-tartrate, disodium L-tartrate, carbon dioxide, lactic acid, sodium lactate, glacial acetic acid, phytic acid, fumaric acid, monosodium fumarate, DL-malic acid, sodium dl-malate, phosphoric acid and the like.

Examples of softeners include glycerol, sorbitol, propylene glycol and the like.

Examples of flavoring agents include menthol, dl-menthol, menthone, vanillin, ethyl vanillin, cinnamic acid, piperonal, d-borneol, maltol, ethyl maltol, camphor, methyl anthranilate, methyl cinnamate, cinnamic alcohol, methyl-N-methylanthranilate, methyl β-naphthyl ketone, limonene, linalool, allyl isothiocyanate and the like.

The chewing composition of the invention is preferably a chewing gum, and more preferably, a sugar-coated gum. Chewing gums can be prepared by, for example, the method and procedures described in Robert Hendrickson, Gum Base and Gum Products Technology and the Great American Chewing Gum Book (CAFOSA GUM S. A., 1974). The Coenzyme Q₁₀ inclusion complex can be added at any stage of the manufacturing process, but is preferably added near the end of the manufacturing process to prevent Coenzyme Q₁₀ from degrading or deteriorating due to heating.

Moreover, in one preferred embodiment of the invention, the chewing composition is coated with sugar, and the sugar coating comprises the Coenzyme Q₁₀ inclusion complex. Specific examples of the chewing composition include foods such as sugar-coated gums, gummies, nougat and the like, in which the core components are coated with sugar; and medicines and quasi drugs such as sugar-coated masticatories, chewable tablets, granules, troches, buccal tablets and the like, in which the core components are coated with sugar. Such sugar-coated products can be prepared by applying over the core, components a sugar coating containing the Coenzyme Q₁₀ inclusion complex according to a usual coating technique. Examples of components of sugar coatings containing the Coenzyme Q₁₀ inclusion complex include, but are not limited to, saccharides such as refined white sugar, glucose, fructose, cane sugar, lactose, maltose, trehalose and the like; sugar alcohols such as erythritol, maltitol, mannitol, sorbitol, xylitol and the like; and polysaccharides such as gum arable, pullulan, hydroxypropylcellulose and the like. One of these saccharides or a mixture thereof is dissolved in water to give a sugar-coating solution, and then the sugar-coating solution is applied over a variety of core components by repeated coating and drying using a coating pan or an automatic sugar coater, thereby preparing the desired sugar-coated products.

The proportion of the Coenzyme Q₁₀ inclusion complex in a sugar coating may be such that the content of the Coenzyme Q₁₀ inclusion complex is 0.01 to 25 wt %, preferably 1 to 10 wt %, and more preferably 2 to 5 wt %, in the final chewing composition (sugar-coated chewing composition). The content can be appropriately set according to the weight ratio between the sugar coating and the core component of the sugar-coated chewing composition. For example, in the case of a sugar-coated chewing composition containing 50 parts by weight of a sugar coating per 100 parts by weight of the core component, the content of the Coenzyme Q₁₀ inclusion complex in 100 wt % of the sugar coating, is 1.5 to 75 wt %, preferably 3 to 30 wt %, and more preferably 6 to 15 wt %.

As will be shown in the Test Examples below, the chewing composition of the invention comprises Coenzyme Q₁₀ in the form of a Coenzyme Q₁₀ inclusion complex with a low Coenzyme Q₁₀ content of 40 wt % or less, preferably 20 wt % or less, and more preferably 10 wt % or less, thereby significantly increasing the efficiency of releasing Coenzyme Q₁₀ in the oral cavity. That is to say, rather than using a small amount of a Coenzyme Q₁₀ inclusion complex with a high content of Coenzyme Q₁₀ of more than 40 wt %, the use of a large amount of a Coenzyme Q10 inclusion complex with a low Coenzyme Q₁₀ content enables a higher intake of Coenzyme Q₁₀, even though the content of Coenzyme Q₁₀ in the final chewing composition is the same in both cases.

Therefore, the chewing composition of the invention is useful in foods containing Coenzyme Q₁₀ which allow efficient, consumption of Coenzyme Q₁₀ during chewing (such as, for example, chewing gums, gummy jellies and nougat which may be sugar-coated); and medicines and quasi drugs containing Coenzyme Q₁₀ (such as, for example, masticatories, chewable tablets, granules, troches, buccal tablets and tooth pastes which may be sugar-coated).

### (2) Method for Improving Release Properties of Coenzyme Q₁₀

The invention also relates to a method for improving the release properties of Coenzyme Q₁₀.

The method can be accomplished by encapsulating Coenzyme Q₁₀ in cyclodextrin. The proportion of Coenzyme Q₁₀ added to cyclodextrin can typically be selected from the range of 1 to 80 wt %; the upper limit being preferably 40 wt %, more preferably 20 wt %, and still more preferably 10 wt %; and the lower limit, being preferably 1 wt %, more preferably 2 wt %, and still more preferably 5 wt %. More specifically, the range is preferably from 1 to 40 wt %, more preferably 2 to 20 wt %, and still more preferably 2 to 10 wt %. The lower the amount of Coenzyme Q₁₀ added to cyclodextrin, i.e., by decreasing it from 40 to 30 wt %, from 30 to 20 wt %, and from 20 to 10 wt %, the better the release properties of Coenzyme Q₁₀ become. The invention was attained based on this finding.

While any of α-, β-, γ- and δ-cyclodextrins are usable in the. invention, β- and γ-cyclodextrins are preferable. γ-Cyclodextrin is more preferable.

The proportion of Coenzyme Q₁₀ to cyclodextrin in the Coenzyme Q₁₀ inclusion complex is not limited in the conditions described hereinabove, but it may be 1 to 80 parts by weight of Coenzyme Q₁₀ to 100 parts by weight of cyclodextrin. The proportion is preferably 2 to 40 parts by weight, and more preferably 5 to 20 parts by weight.

The method of the invention improves the release properties of Coenzyme Q₁₀ in the oral, cavity. The method is thus suitably used especially for the preparation of compositions chewed in the mouth, or compositions consumed by chewing them (i.e., chewable compositions); for example, foods such as chewing gums, gum jellies, nougat and the like, and medicines and quasi, drugs such as masticatories, chewable tablets, granules, troches, buccal tablets, tooth pastes and the like. Hence, the invention provides a method for improving the release properties of Coenzyme Q₁₀ in the oral cavity from a chewing composition containing Coenzyme Q₁₀.

The method involves the use of a composition comprising Coenzyme Q₁₀ that is encapsulated in cyclodextrin (a Coenzyme Q₁₀ inclusion complex) as a Coenzyme Q₁₀ component. As previously described, the amount of Coenzyme Q₁₀ added to cyclodextrin can typically be selected from the range of 1 to 80 wt %; the upper limit being preferably 40 wt %, more preferably 20 wt %, and still more preferably 10 wt %; and the lower limit being preferably 1 wt %, more preferably 2 wt %, and still more preferably 5 wt %. More specifically, the range is preferably from 1 to 40 wt %, more preferably 2 to 20 wt %, and still more preferably 2 to 10 wt %.

The proportion of Coenzyme Q₁₀ to cyclodextrin in the Coenzyme Q₁₀ inclusion complex is also as described above, hence being 1 to 80 parts by weight, preferably 2 to 40 parts by weight, and more preferably 5 to 20 parts by weight, per 100 parts by weight of cyclodextrin.

In the method of the invention, conventional ingredients suitable to the type and shape of the chewing composition, in addition to the Coenzyme Q₁₀ inclusion complex, can be added to the chewing composition, as long as the Coenzyme Q₁₀ inclusion complex is used as a Coenzyme Q₁₀ component. While the proportion of the Coenzyme Q₁₀ inclusion complex in the chewing composition is not limited, it may suitably be selected from the typical range of 0.08 to 25 wt % per 100 wt % of the chewing composition. The range is preferably from 1 to 10 wt %, and more preferably 2 to 5 wt %. The proportion of the aforementioned range is preferably adjusted such that Coenzyme Q₁₀ is contained in an amount of typically 0.01 to 10 wt %, preferably 0.1 to 4 wt %, and more preferably 0.2 to 2 wt %, per 100 wt % of the chewing composition.

Moreover, in the method of the invention, the chewing composition may be covered with a sugar coating containing the Coenzyme Q₁₀ inclusion complex. The processes for preparing such a sugar coating and sugar-coated chewing composition, as well as the content of the Coenzyme Q₁₀ inclusion complex in the sugar coating are the same as described in Section (1) above, whose description is herein also useful.

### Examples

The invention will be described below in detail with reference to the Test Examples shown below, which are not intended to limit the invention. In the following formulations, “part” or “parts” denotes “part(s) by weight”, and “%” denotes “% by weight”, unless otherwise stated. As used hereinbelow, the symbol “*” indicates a product manufactured by San-Ei Gen F. F. I., Inc; and the symbol “^{‡}” indicates a trademark of San-Ei Gen F. F. I., Inc.

### Test Examples

### Test Example 1

### Preparation of Samples Containing Coenzyme Q₁₀

Samples containing Coenzyme Q₁₀ (Samples 1, 2, 3 and 4) were prepared according to the formulations shown in Table 1. For comparison, neat Coenzyme Q₁₀ was used as Sample 5.

TABLE 1

| | Sample 1 | Sample 2 | Sample 3 | Sample 4 | Sample 5 |
|---|---|---|---|---|---|
| Coenzyme | 10 | 20 | 40 | 40 | 100 |
| Q₁₀ | | | | | |
| γ- | 90 | 80 | 60 | — | — |
| Cyclodextrin | | | | | |
| Gum Arabic | — | — | — | 30 | — |
| Dextrin | — | — | — | 30 | — |
| Total | 100 | 100 | 100 | 100 | 100 |

(Units: % by weight)

### (1-1) Preparation of Sample 1

In 3000 parts of water, 270 parts of γ-cyclodextrin were dissolved by heating and maintained at 70° C. Thirty parts of Coenzyme Q₁₀ were melted by heating and added to 3270 parts of the solution obtained above, and stirred at 70° C. for 24 hours. The resulting solution was spray dried to give 290 parts of a powder (Sample 1, Coenzyme Q₁₀:γ-cyclodextrin=10:90 by weight).

### (1-2) Preparation of Sample 2

The same procedures as in Preparation (1-1) were repeated, except that 240 parts of γ-cyclodextrin, 3000 parts of water, and 60 parts of Coenzyme Q₁₀ were used, so as to give 290 parts of a powder (Sample 2, Coenzyme Q₁₀:γ-cyclodextrin=20:80 by weight).

### (1-3) Preparation of Sample 3

The same procedures as in Preparation (1-1) were repeated, except that 180 parts of γ-cyclodextrin, 3000 parts of water, and 40 parts of Coenzyme Q₁₀ were used, so as to give 290 parts of a powder (Sample 3, Coenzyme Q₁₀:γ-cyclodextrin=40:60 by weight).

(1-4) Preparation of Sample 4

In 700 parts of water, 90 parts of gum arable and 90 parts of dextrin were dissolved by heating and maintained at 70° C. To 880 parts of the resulting solution were added 120 parts of Coenzyme Q₁₀ melted by heating, and stirred at 70° C. for 1 hour. The resulting solution was emulsified at 400 kg/cm² with a homogenizer, and spray dried to give 290 parts of a powder (Sample 4, Coenzyme Q₁₀:gum arabic:dextrin=40:30:30 by weight).

### (1-5) Preparation of Sample 5

A Coenzyme Q₁₀ crystalline powder (manufactured by San-Ei Gen F. F. I., Inc.) was used as Coenzyme Q₁₀ of Sample 5.

### (2) Preparation of Stick Gums

Using one of Samples 1 to 5 obtained above, raw materials were blended according to the formulation shown below such that the content of Coenzyme Q₁₀ in 100 g of the resulting stick gum would be 48 mg, and each mixture was molded to form a stick gum. Specific procedures are as follows: A gum base was first kneaded at 70° C. with sugar, corn syrup and glycerol, and the mixture was blended with Lemon Oil No. 2451* (flavoring agent) and San Yellow^{‡} No. 3* (colorant). One each of Samples 1, 2, 3, 4 or 5 was then added to the resulting mixture. After cooling to room temperature, each material was rolled and molded to prepare a chewing gum (stick gum). The gum base used in the following formulation was purchased from Lotte, Co., Ltd. (Gum Base LT1).

Gum Formulation

| Samples 1-5 | See Table 2. | |
|---|---|---|
| | (wt %) | |
| Lemon Oil No. 2451* | 1.0 | |
| San Yellow^{‡} No. 3* | 0.1 | |
| Sugar | 62.0 | |
| Corn syrup | 10.0 | |
| Glycerol | 1.0 | |
| Gum base | Remainder | |
| Total | 100.0 | wt %. |

Table 2 shows the amounts of Coenzyme Q₁₀ (CoQ₁₀ Content) contained in Samples 1 to 5, the contents of Samples 1 to 5 in the stick gums (Sample Content), and the CoQ₁₀ contents in the stick gums (all the contents are expressed in wt %).

TABLE 2

| | CoQ₁₀ Content | | CoQ₁₀ Content |
|---|---|---|---|
| Sample | in Sample | Sample Content | in Gum |
| Sample 1 | 9.7 | 0.495 | 0.048 |
| Sample 2 | 21.4 | 0.224 | 0.048 |
| Sample 3 | 42.0 | 0.115 | 0.048 |
| Sample 4 | 42.2 | 0.114 | 0.048 |
| Sample 5 | 100.0 | 0.048 | 0.048 |

(Units: % by weight)

### (3) Analysis of Coenzyme Content

### (3-1) Analysis of Coenzyme Q₁₀ Contents in Gums

Each type of the stick gums thus prepared was sampled in a precise amount of 4 g, and placed in a stoppered centrifuge tube. To the tube was added 10 ml of a 20% (v/v) tetrahydrofuran aqueous solution. The tube was then equipped with a Dimroth condenser, and the solution was refluxed, while occasionally mixing it by shaking, in a hot bath at 82° C. for 30 minutes. The refluxed solution was then cooled to room temperature, and 15 ml of diethyl ether was added, followed by extraction with shaking for 10 minutes. The extract was centrifuged at 4000 rpm for 3 minutes, and then the upper layer (diethyl ether layer) was poured into a 50-ml volumetric flask, and 10 ml of diethyl ether was added to the remaining bottom layer, after which the mixture was subjected to shaking for 10 minutes and centrifuged (extraction operation). This extraction operation was repeated a total of three times, each of the upper layer (diethyl ether layer) was poured into the above flask, and lastly, the flask was filled with diethyl ether to 50 ml. To three milliliters of this solution, anhydrous ethanol was added to 50 ml. The solution was then filtered with a 0.45-μm PTFE filter, and the recovered filtrate was used as an analytical sample. Each of the samples was subjected to HPLC under the conditions described below to determine the Coenzyme. Q₁₀ content in the gum (CoQ₁₀ Content in Gum). The results shown in Table 2 confirmed that the content of Coenzyme Q₁₀ in each of the stick gums was 0.048 wt %.

(3-2) Analysis of Release of Coenzyme Q₁₀ into Saliva

Each type of the gums prepared above were sampled in a precise amount of 1 g (total 4 g for four individuals), and then 1 g each of these gums was chewed by four subjects under the following conditions. The gums chewed by the four subjects were each placed in a stoppered centrifuge tube. The same procedures as before chewing (see (3-1) above) were thereafter repeated for extraction, and each recovered filtrate was used as a sample for HPLC analysis to measure the Coenzyme Q₁₀ content in the chewed gum (CoQ₁₀ Content in Chewed Gum).

Chewing Conditions
Number of subjects: Four
Chowing time: 5 min
Chowing frequency: 60 times/min (once per second)
Chowing amount: 1 g

The proportion of Coenzyme Q₁₀ released into saliva was determined based on Equation 1:

$$Release of Coenzyme {Q}_{10} into saliva ( \% ) = \frac{( \begin{matrix}{CoQ}_{10} Content in Gum - \\ {CoQ}_{10} Content in Chewed Gum\end{matrix} )}{{CoQ}_{10} Content in Gum} \times 100 \qquad [ Equation 1 ]$$

### (3-3) HPLC Analysis

HPLC analysis was performed under the following conditions:
Column: Wakosil-II 5C₁₈HG (Wako Pure Chemical Ind. Ltd.), 4.6×250 mm
Column temperature: 35° C.
Mobile phase: methanol/ethanol=12/8
Flow rate: 2.0 ml/min
Detection wavelength: UV 275 nm (maximum wavelength of Coenzyme Q₁₀)
Injected amount: 60 μl

A Coenzyme Q₁₀ standard solution for HPLC (standard solution) was prepared as follows.

### Coenzyme Q₁₀ Standard Solution

Coenzyme Q₁₀ (Wako Pure Chemical Ind. Ltd.) was taken precisely in an amount of 0.05 g, and a small amount of anhydrous ethanol was added and heated to 50° C. to completely dissolve the Coenzyme Q₁₀. After cooling to room temperature, anhydrous ethanol was added to 50 ml, and the resulting solution was diluted with anhydrous ethanol to a predetermined concentration. This solution was used as a Coenzyme Q₁₀ standard solution for HPLC.

### (4) Results

Table 3 shows the results obtained by examining the proportions of Coenzyme Q₁₀ released into saliva from the gums prepared using Samples 1 to 5 according to the above-described procedures.

TABLE 3

| Sample | Release (%) |
|---|---|
| Sample 1 | 33.5 |
| Sample 2 | 13.3 |
| Sample 3 | 11.5 |
| Sample 4 | 8.2 |
| Sample 5 | 0 |

The results show that the release of Coenzyme Q₁₀ into saliva was improved by using Samples 1 to 4 containing Coenzyme Q₁₀; in contrast, 0% of the Coenzyme Q₁₀ was released into saliva from the gum prepared using Sample 5 (neat CoQ₁₀). Moreover, the results for Samples 1 to 4 show that the release of Coenzyme Q₁₀into saliva was improved by encapsulating Coenzyme Q₁₀ in cyclodextrin (Samples 1 to 3). In addition, comparisons of Samples 1 to 3 show that the smaller the content of Coenzyme Q₁₀, the greater the release of Coenzyme Q₁₀ into saliva becomes. The foregoing findings show that in order to allow efficient release of a large amount of Coenzyme Q₁₀ from a gum, it is preferable to use a large amount of a Coenzyme Q₁₀ inclusion complex with a low content of Coenzyme Q₁₀, rather than using a Coenzyme Q₁₀ inclusion complex with a high content of Coenzyme Q₁₀.

### Test Example 2

### Preparation of Sugar-Coated Gums

Sugar-coated gums each comprising a sample containing Coenzyme Q₁₀ in the sugar coating were prepared, and the release of Coenzyme Q₁₀ into saliva from these gums was evaluated. More specifically, sugar coatings were prepared using, as a sample containing Coenzyme Q₁₀, Sample 1, 2, 4 or 5 as set forth in Test Example 1, or Sample 6 of the composition shown in Table 4. Using these sugar coatings, sugar-coated tablet gums were prepared.

### (1) Preparation of Sample Containing Coenzyme Q₁₀ (Sample 6)

A sample containing Coenzyme Q₁₀ (Sample 6) was prepared according to the formulation shown in Table 4. More specifically, 90 parts of gum arable and 150 parts of dextrin were dissolved in 700 parts of water by heating, and maintained at 70° C. Sixty parts of Coenzyme Q₁₀ were melted by heating and added to 880 parts of the solution obtained above, and stirred at 70° C. for 1 hour. The resulting solution was emulsified at 400 kg/cm² with a homogenizer, and spray dried to give 290 parts of a powder (Sample 6, Coenzyme Q₁₀:gum arabic:dextrin=20:30:50 by weight).

TABLE 4

| | Sample 6 |
|---|---|
| Coenzyme Q10 | 20 |
| Gum Arabic | 30 |
| Dextrin | 50 |
| Total | 100 |

(Units: % by weight)

### (2) Preparation of Sugar-Coated Gums

Sugar-coated gums (1.5 g/tablet) each comprising Coenzyme Q₁₀ in the sugar coating were prepared in the manner described below such that the content of Coenzyme Q₁₀ in 100 g of the sugar-coated gum was 20 mg.

### (2-1) Preparation of Gum Centers

A gum base (trade name: Gum Base LT2, manufactured by Lotte, Co., Ltd.) was kneaded at 70° C. with xylitol, corn syrup and glycerol, and after the addition of Lemon Oil No. 2451* (flavoring agent), the mixture was cooled to room temperature and molded to a tablet shape.

Formulation of Gum Centers (wt %)

| Xylitol | 30 | |
|---|---|---|
| Corn syrup | 5 | |
| Glycerol | 1 | |
| Lemon Oil No. 2451* | 1 | |
| Gum base | Remainder | |
| Total | 100.0 | wt %. |

### (2-2) Preparation of Sugar Coatings

Maltitol and gum arabic were added to water and dissolved by heating. After cooling the mixture to room temperature, one each of Sample 1, 2, 4, 5 or 6 was added and mixed homogeneously.

Formulation of Sugar Coatings

| Sample 1, 2, 4, 5 or 6 | see Table 5 | |
|---|---|---|
| | (wt %). | |
| Maltitol | 70.0 | |
| Gum Arabic | 3.0 | |
| Water | Remainder | |
| Total | 100.0 | wt %. |

The sugar solutions thus obtained were each spray dried over the gum centers using a coating pan and dried. The proportion of the sugar-coating layer was 50 parts by weight to 100 parts by weight of the gum center.

Table 5 shows the amounts of Coenzyme Q₁₀ (CoQ₁₀ Content) contained in Samples 1, 2 and 4 to 6, the contents of the Samples in the sugar coatings, the contents of the Samples in the sugar-coated gums, and the CoQ₁₀ contents in the sugar-coated gums (all the contents are expressed in wt %).

TABLE 5

| | | Sample | Sample | CoQ₁₀ |
|---|---|---|---|---|
| | CoQ₁₀ | Content in | Content in | Content in |
| | Content in | Sugar | Sugar- | Sugar- |
| Sample | Sample | Coating | Coated Gum | Coated Gum |
| Sample 1 | 9.7 | 0.618 | 0.206 | 0.020 |
| Sample 2 | 21.4 | 0.279 | 0.093 | 0.020 |
| Sample 4 | 42.2 | 0.141 | 0.047 | 0.020 |
| Sample 5 | 100.0 | 0.060 | 0.020 | 0.020 |
| Sample 6 | 20.0 | 0.300 | 0.100 | 0.020 |

(Units: % by weight)

### (3) Analysis of Coenzyme Content

### (3-1) Analysis of Coenzyme Q₁₀ Contents in Gums

Each type of the sugar-coated gums prepared above were sampled in a precise amount of 6 g (total weight of four pieces), and placed in a stoppered centrifuge tube. The same procedures as in Test. Example 1 were performed to prepare sample filtrates for HPLC analysis. Each of the samples was subjected to HPLC under the same conditions as in Test Example 1 to determine the Coenzyme Q₁₀ content in the sugar-coated gum (CoQ₁₀ Content in Sugar-Coated Gum). The results shown in Table 5 confirmed that the content of Coenzyme Q₁₀ in each type of sugar-coated gums was 0.02 wt %.

(3-2) Analysis of Release of Coenzyme Q₁₀ into Saliva

Each type of the sugar-coated gums prepared above were sampled in a precise amount of 6 g (total weight of four pieces), and one piece each of these gums was chewed by four subjects under the same conditions as in Test Example 1. The gums chewed by the four subjects were each placed in a stoppered centrifuge tube. The same procedures as in Test Example 1 were thereafter repeated, and the resulting filtrates were used as samples for HPLC analysis to measure the Coenzyme Q₁₀ contents in the chewed gums (CoQ₁₀ Content in Chewed Gum). The samples were subjected to HPLC under the same conditions to determine the contents of Coenzyme Q₁₀ in the chewed gums, and then the release of Coenzyme Q₁₀ into saliva from these gums was evaluated as in Test Example 1.

### (4) Results

Table 6 shows the results obtained by examining the proportions of Coenzyme Q₁₀ released into saliva from the gums prepared using Samples 1, 2, 4, 5 or 6.

TABLE 6

| Sample | Release (%) |
|---|---|
| Sample 1 | 100.0 |
| Sample 2 | 94.7 |
| Sample 4 | 50.2 |
| Sample 5 | 30.0 |
| Sample 6 | 48.3 |

The results show that the gums prepared using Samples 1 and 2 released about 100% and about 95% of the Coenzyme Q₁₀, respectively, which were remarkably higher than about 50%, 30% and 50% from the gums prepared using Samples 4, 5 and 6, respectively. This finding shows that the release properties of Coenzyme Q₁₀ are improved by encapsulating Coenzyme Q₁₀, and in particular, a lower amount of Coenzyme Q₁₀, in cyclodextrin.

### Example 1 to 3

Using Samples 1 to 5 containing Coenzyme Q₁₀ as prepared in Test Examples, sugar-coated gums were prepared in the manner described below.

### (1) Preparation of Sugar-Coated Gums

Using Sample 1 containing Coenzyme Q₁₀, sugar-coated gums comprising Coenzyme Q₁₀ in the sugar coating (1.5 g/piece) were prepared according to the following formulation such that the content of Coenzyme Q₁₀ was 20 mg in 100 g of the sugar-coated gum.

### (1-1) Preparation of Gum Centers

A gum base (trade name: Gum Base LT2, manufactured by Lotte, Co., Ltd.) was kneaded at 70° C. with corn syrup and glycerol, and after the addition of Lemon Oil No. 2451* (flavoring agent), the mixture was cooled to room temperature and molded to a tablet shape.

Formulation of Gum Centers (wt %)

| | |
|---|---|
| Corn syrup | 5 |
| Glycerol | 1 |
| Lemon Oil No. 2451* | 1 |
| Gum Base | Remainder |
| Total | 100.0 |

### (1-2) Preparation of Sugar Coatings

Maltitol and gum arable were added to water and dissolved by heating. After cooling the mixture to room temperature, one each of Samples 1, 4 or 5 was added and mixed homogeneously.

Formulation of Sugar Coatings (wt %)

| Sample 1, 4 or 5 | see Table 7 |
|---|---|
| Maltitol | 70.0 |
| Gum Arabic | 3.0 |
| Water | Remainder |
| Total | 100.0. |

The sugar-coating solutions thus obtained were each spray dried over the gum centers using a coating pan and dried. The proportion of the sugar-coating layer was 50 parts by weight to 100 parts by weight of the gum center.

TABLE 7

| | | Sample Content in |
|---|---|---|
| Example | Sample Used | Sugar-Coated Gum (wt %) |
| 1 | Sample 1 | 0.206 |
| 2 | Sample 4 | 0.047 |
| 3 | Sample 5 | 0.020 |

As shown in Table 7, the sugar-coated gum comprising Sample 1 with the lowest Coenzyme Q₁₀ content exhibited the highest release properties of Coenzyme Q₁₀. Moreover, samples were prepared in the same manner as in Examples 1, 2 or 3, except that a sugar or sugar alcohol was added to Gum Base LT2 (manufactured by Lotte., Co., Ltd.), and the release of Coenzyme Q₁₀ from these samples was similarly measured. The results showed that as in the experimentation shown above, regardless of the use of a sugar or a sugar alcohol, the sugar-coated gum comprising Sample 1 with the lowest Coenzyme Q₁₀ content exhibited the highest release properties of Coenzyme Q₁₀.

1. A chewing composition comprising a Coenzyme Q₁₀ inclusion complex in which Coenzyme Q₁₀ is encapsulated in cyclodextrin.

2. The chewing composition according to claim 1, wherein the Coenzyme Q₁₀ inclusion complex comprises 1 to 40 wt % of Coenzyme Q₁₀.

3. The chewing composition according to claim 1, wherein the content of Coenzyme Q₁₀ is 1 to 80 parts by weight to 100 parts by weight of cyclodextrin.

4. The chewing composition according to claim 1, wherein the content of the Coenzyme Q₁₀ inclusion complex is 0.08 to 25 wt % in 100 wt % of the chewing composition.

5. The chewing composition according to claim 1, wherein the content of the Coenzyme Q₁₀ inclusion complex is such that the proportion of Coenzyme Q₁₀ in 100 wt % of the chewing composition is 0.01 to 10 wt %.

6. The chewing composition according to claim 1, wherein the cyclodextrin is γ-cyclodextrin.

7. The chewing composition according to claim 1, further comprising a gum base.

8. The chewing composition according to claim 7, which is a chewing gum.

9. The chewing composition according to claim 1, wherein the chewing composition is covered with a sugar coating comprising the Coenzyme Q₁₀ inclusion complex.

10. A method for improving the release properties of Coenzyme Q₁₀, comprising encapsulating Coenzyme Q₁₀ in cyclodextrin.

11. A method for improving the release properties of Coenzyme Q₁₀ from a chewing composition containing Coenzyme Q₁₀, the method comprising adding to the composition a Coenzyme Q₁₀ inclusion complex in which Coenzyme Q₁₀ is encapsulated in cyclodextrin as a Coenzyme Q₁₀ component.

12. The method according to claim 11, wherein the Coenzyme Q₁₀ inclusion complex comprises 1 to 40 wt % of Coenzyme Q₁₀.

13. The method according to claim 11, wherein the content of Coenzyme Q₁₀ in the Coenzyme Q₁₀ inclusion complex is 1 to 80 parts by weight to 100 parts by weight of cyclodextrin.

14. The method according to claim 11, wherein the content of the Coenzyme Q₁₀ inclusion complex is 0.08 to 25 wt % in 100 wt % of the chewing composition.

15. The method according to claim 11, comprising adding to the composition the Coenzyme Q₁₀ inclusion complex in an amount such that the content of Coenzyme Q₁₀ is 0.01 to 10 wt % in 100 wt % of the chewing composition.

16. The method according to claim 11, wherein the cyclodextrin is γ-cyclodextrin.

17. The method according to claim 11, wherein the chewing composition further comprises a gum base.

18. The method according to claim 17, wherein the chewing composition is a chewing gum.

19. The method according to claim 11, wherein the chewing composition is covered with a sugar coating comprising the Coenzyme Q₁₀ inclusion complex.

20. The method according to claim 11, which is for improving the release properties of Coenzyme Q₁₀ in the oral cavity from a chewing composition containing Coenzyme Q₁₀.

21. Use of a Coenzyme Q₁₀ inclusion complex in which Coenzyme Q₁₀ is encapsulated in cyclodextrin, for the preparation of a chewing composition containing Coenzyme Q₁₀.

22. The use according to claim 21, wherein the chewing composition is a chewing gum.
